(19) United States
(12) Patent Application Publication
(10) Pub. No.: US 2024/0108500 A1
(43) Pub. Date: Apr. 4, 2024

(54) IMPLANT

(71) Applicant: Inflammasome Therapeutics, Inc., Newton, MA (US)

(72) Inventors: Paul Ashton, Newton, MA (US); Hong Guo, Wayland, MA (US); Jianbing Chen, Wayland, MA (US)

(21) Appl. No.: 18/375,713

(22) Filed: Oct. 2, 2023

Related U.S. Application Data
(60) Provisional application No. 63/412,678, filed on Oct. 3, 2022.

(51) Int. Cl. A61F 9/00; A61L 27/16; A61L 27/54; A61L 27/58

(52) U.S. Cl. CPC A61F 9/0017; A61L 27/16; A61L 27/54; A61L 27/58; A61F 2250/0067; A61L 2300/204; A61L 2300/232

(57) ABSTRACT

A drug delivery implant may include an implant body and a core comprising a pharmaceutical agent. The core can be located within the implant body that extends longitudinally and circumferentially around the core. The implant body can also define an aperture that exposes a first surface of the core. A method for treating a mammal to obtain a desired physiological or pharmacological effect by injecting such a drug delivery implant into a mammal in need of treatment is also disclosed.

### CROSS REFERENCE TO RELATED APPLICATIONS

This application claims the benefit of U.S. Provisional Patent Application No. 63/412,678, filed Oct. 3, 2022; the entire contents of which are incorporated herein by reference.

### BACKGROUND

In the treatment of eye diseases, injections are usually preferred to systemic drug administration because the blood/retinal barrier impedes the passage of most drugs from the circulating blood to the interior of the eye, and local administration to the eye circumvents any off-target effects of the drug in other parts of the body. There are many diseases of the eye that require constant administration of a bioactive agent, either life-long such as in the treatment of glaucoma or in retinal disorders, or for prolonged periods (months) such as after surgery. However, the half-life of most injected compounds in the vitreous, especially small drug molecules, is relatively short, usually on the scale of just a few hours. As a result, intravitreal injection treatments typically require frequent administration, which is undesirable due to the associated pain, discomfort, intraocular pressure increases, intraocular bleeding, increased chances for infection, and the possibility of retinal detachment. Thus, there is still a need for devices and methods that support the controlled, sustained, local administration of a drug to the eye.

There is also a need for injectable, removable, bioerodible sustained release devices that can maintain steady state drug levels systemically. There are many examples of injectable bioerodible drug delivery systems that provide non-linear release (e.g., PLGA, microspheres, liposomes etc.) but these typically provide varied plasma levels (initially very high, declining in a logarithmic fashion) and cannot be easily removed. Alternatively, there are non-erodible implants (Jadelle, Norplant, etc.) that provide reasonable sustained release kinetics but as they become depleted the release rate slowly falls giving a long “tail-off” period and they must be removed when plasma levels fall below a desired range.

### SUMMARY OF THE INVENTION

In certain embodiments, the present disclosure provides a drug delivery implant shaped and sized for injection. The drug delivery implant can comprise a core comprising a pharmaceutical agent. The drug delivery implant can also comprise a cylindrical implant body that is configured to extend longitudinally around and circumferentially contact a longitudinal surface of the core. The implant body can define an aperture at a first end of the implant body to expose a first surface of the core. When placed in an aqueous environment, the pharmaceutical agent can diffuse from the first surface of the core through the aperture at the first end of the implant body. In some embodiments, the pharmaceutical agent has a solubility less than about 2 mg/ml.

In certain embodiments, the present disclosure provides a drug implant comprising a core. The core can comprise a pharmaceutical agent having a solubility less than about 2 mg/ml.

In certain embodiments, the present disclosure provides a method for treating a subject in need thereof comprising implanting an implant according to the present disclosure.

Numerous embodiments are further provided that can be applied to any aspect of the present disclosure as described herein.

In certain embodiments the release rate of drug is maintained at a relatively constant level until it becomes almost entirely depleted. The “tail-off” period is consequently very short.

### BRIEF DESCRIPTIONS OF THE DRAWINGS

FIGS. 1A-B show a system (FIG. 1A) comprising an implant body a core comprising a pharmaceutical agent showing that drug release is relatively constant after an initial burst phase (FIG. 1B).

FIG. 2 shows that steady state release is proportional to the surface area of the core.

FIG. 3 shows that the surface area of the core is substantially constant until depletion of the pharmaceutical agent.

FIG. 4 shows that the release rate of the pharmaceutical agent is substantially constant until depletion.

FIGS. 5A-B shows that release rate/duration of the pharmaceutical agent is independent of the composition of the implant body. FIG. 5A shows release of K8 (2-Et-AZT) from an implant body comprising polyimide (non-erodible) (ID=0.34 mm and 3.5 mm long (n=6); drug loading ˜350 μg, duration ˜120 days). FIG. 5B shows cumulative amount of K8 released from an implant body comprising PLGA (ID=0.35 mm and 3.5 mm long (n=4); drug loading ˜380 μg, duration ˜125 days).

FIGS. 6A-B show that duration of pharmaceutical agent release is dependent on length. FIG. 6A shows a 2.5 mm long tube containing 320 μg K8 provides sustained release for ˜60 days (ID=0.37 mm and 2.5 mm long (n=4). FIG. 6B shows a 5 mm long tube containing 610 μg K8 provides sustained release for ˜220 days (polyimide body (ID=0.37 mm and 5 mm long (n=4).

FIG. 7 shows cumulative release of K8 from an implant (ID=0.37 mm) in-vitro compared to in-vivo.

FIG. 8 shows release of the pharmaceutical agent EFdA (islatravir) from an implant occurs over ˜100 days.

FIG. 9 shows the release profile of islatravir from an implant over ˜120 days. If the release surface area is equivalent to 3.14 mm², the daily release rate at steady state phase is around 75 μg/day. Implants in PLGA tube (bio-degradable) showed similar in vitro release profile compared to in polyimide tube (non-degradable).

FIG. 10 shows daily release rate of the pharmaceutical agent before depletion. The implants showed a short tail and depleted completely in a few days in vitro.

FIG. 11 shows batch sample results for EFdA Plasma Concentration in Rats up to one month.

### DETAILED DESCRIPTION OF THE INVENTION

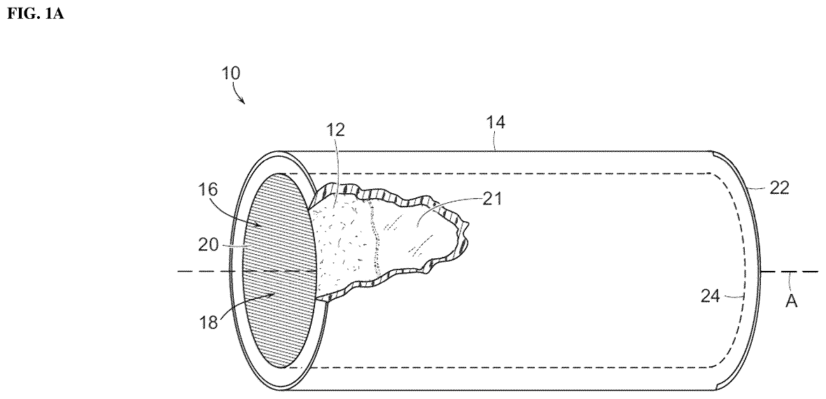

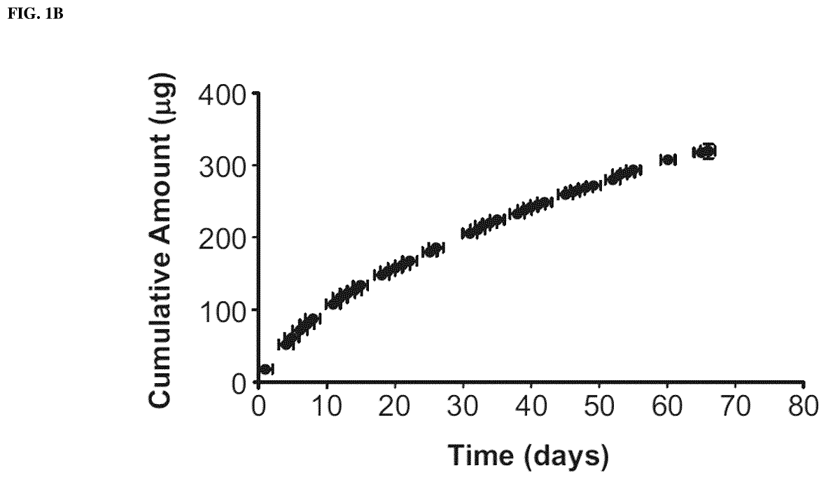

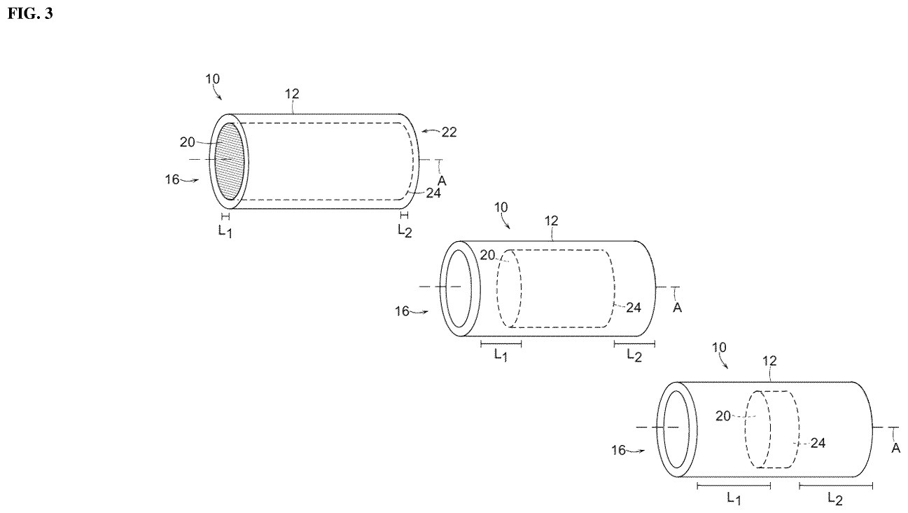

The implants described herein are suitable for the controlled and sustained release of moderate-low solubility or low solubility drugs. A drug delivery implant 10 comprises a core 12 comprising an effective amount of a pharmaceutical agent (e.g., a moderate-low solubility or low solubility drug). The core 12 is located within a cylindrical implant body 14 that is arranged to extend around the core 12 along axis A1, as shown in FIG. 1. The implant body 14 defines an aperture 16, for example at at a first end 18 of the implant body 14, to expose a first surface 20 of the core 12, as shown in FIG. 1. When the implant 10 is placed in an aqueous environment, the pharmaceutical agent diffuses from the first surface 20 through the aperture 16 at the first end 18 of the implant body 14, as shown in FIGS. 1 and 3. In certain preferred embodiments, the implant 10 does not contain a rate-controlling membrane that covers the aperture 16; the first surface is preferably in direct contact with the surrounding environment.

In certain embodiments the implant body 14 is impermeable to, or substantially impermeable to, the passage of water and impermeable to, or substantially impermeable to, diffusion of the pharmaceutical agent in the core so that pharmaceutical agent release only, or primarily, occurs via the aperture.

In certain embodiments the implant body 14 is biodegradable and remains impermeable or substantially impermeable to water and diffusion of the pharmaceutical agent until the pharmaceutical agent in the core has been substantially or entirely released after which time the implant body 14 biodegrades.

In certain embodiments, the first surface 20 has an initial surface that is coterminous with the aperture 16, as shown in FIG. 1. In certain preferred embodiments, the first end 18 of the implant body 14 is not capped. For example, when the implant 10 is implanted or placed in an aqueous environment, the first surface 20 is directly exposed to the aqueous environment along the entirety of the first surface 20. While in the aqueous environment, the area of the exposed surface remains substantially constant as the core 12 dissolves and allows the pharmaceutical agent to diffuse through the aperture 16.

In certain embodiments, the core 12 comprises drug particles and the pharmaceutical agent is present in the drug particles. In certain embodiments, a core comprises a pellet (not shown) that comprises the drug particles and can optionally contain a polymeric material (e.g., polyvinyl alcohol) that does not substantially affect the release of the pharmaceutical agent. When present, the polymeric material can maintain the available surface area of the core 12.

In certain embodiments, the core 12 comprises a matrix that comprises the pharmaceutical agent and a polymeric material such that the pharmaceutical agent is dispersed through the matrix. When present in the core 12 as part of the matrix or the pellet, the polymeric material is present at less than about 10% w/w of the core, less than about 5% w/w of the core, or less than about 2% w/w of the core. In certain embodiments, the polymeric material is present at about 2.5% w/w of the core. In certain embodiments, the polymeric material does not dissolve in the aqueous environment and maintains the available surface area of the core 12. In certain preferred embodiments, the polymeric material does not substantially affect the rate of the release of the pharmaceutical agent. In certain embodiments, the polymeric material bioerodes. In certain embodiments, the polymeric material is PVA.

In certain embodiments, the core 12 comprises a layer 21 of polymeric material that forms a longitudinal surface of the core 12, as shown in FIG. 1. In some embodiments, the coating at least partially surrounds the core 12, or even completely surrounds the core 12. In some embodiments, the coating comprises a second polymer, e.g., a hydrophilic polymer, such as PVA. In certain embodiments, the PVA of the coating has a molecular weight from about 50 KDa to about 300 KDa. In some embodiments, the PVA of the coating has a degree of hydrolysis from about 80% to about 98%.

For example, the core 12 can include a layer 21 of polymeric material that is located between the implant body 14 and the pharmaceutical agent. In certain embodiments, the polymeric material is PVA. In some embodiments, the core comprises a compressed pellet of pharmaceutical agent and the compressed pellet is dipped in a solution of polymeric material (e.g., PVA) to form the layer 21 before being inserted into the implant body 14. In certain embodiments, the core 12 does not include the layer such that the pharmaceutical agent directly contacts the implant body 12.

The implant body 14 surrounds the core 12, as shown in FIG. 1, and is substantially impermeable to water. In some embodiments, the implant body is non-bioerodible and comprises polyimide, for example. In some embodiments, the implant body is bioerodible and comprises PLGA, PCL, or PLA, for example. Polymers such as PLGA are available in different erosion rates. Polymers such a PLGA, which undergo “bulk” erosion are well suited to these systems. In some embodiments, the implant body does not erode until the drug has been released.

The implant body 14 includes a second end 22 that is spaced apart from the first end 18, as shown in FIG. 1. The implant body 14 defines a second aperture 24 located at the second end 22 of the implant body 14. In some embodiments, the second end is capped (not shown) so that only the first surface 20 is exposed in the aqueous environment. In preferred embodiments, the cap is impermeable or substantially impermeable. In some embodiments, the cap comprises the same material as the implant body 14. In some embodiments, the implant body defines an aperture along the longitudinal surface. In some embodiments both ends of the implant body are capped and an aperture is made in the wall (i.e., a longitudinal surface) of the implant body through which drug is released. In some embodiments, the implant body defines more than one aperture, for example at a first end, a second end, one or more along the longitudinal surface, and combinations thereof. In some embodiments, two or more apertures on the longitudinal surface expose surfaces of the core. In some embodiments, the apertures are not covered with a rate-controlling membrane; preferably, the exposed surfaces are in direct contact with the surrounding environment.

In some embodiments, the second end 22 is not capped so that the second aperture 24 exposes a second surface 26 of the core 12, which is coterminous with the aperture 24, as shown in FIG. 1. In some embodiments, the surface area of the second surface 26 is substantially the same as the surface area of the first surface 20. When placed in an aqueous environment, the pharmaceutical agent can diffuse out of the first aperture 16 and the second aperture 24.

In certain embodiments, the exposed surface area is relatively constant as the core 12 releases about 10% to about 90% or about 15% to about 80% of the pharmaceutical agent. In certain embodiments, the exposed surface area is relatively constant as the core 12 releases up to about 80%, up to about 85%, up to about 90%, up to about 95%, or up to about 99% of the pharmaceutical agent. In certain embodiments, the exposed surface of the core (e.g., first surface 20 or second surface 26) remains substantially constant. For example, the exposed surface area can be relatively constant as the core releases about 10% to about 90% of the pharmaceutical agent. In certain embodiments, the surface area is about 0.05 mm² to about 0.3 mm² or about 0.06 mm² to about 0.21 mm².

As the pharmaceutical agent diffuses out of the aperture 16, the first surface 20 of the core 12 recedes a distance L1 from the first aperture 16, as shown in the middle and bottom panels of FIG. 3. As the pharmaceutical agent diffuses out of the aperture 24, the second surface 26 of the core 12 recedes a distance L2 from the aperture 24, as shown in the middle and bottom panels of FIG. 3. When in the aqueous environment, L1 is substantially the same as L2. Alternatively, if the second surface 26 is capped, L1 is substantially greater than L2 when the implant 10 is in an aqueous environment.

The implant 10 may be implanted for a sufficient period of time and under conditions to allow treatment of the disease state of concern. For example, the implant 10 can release the pharmaceutical agent for at least a month, at least two months, at least three months, or at least six months, or at least 12 months, or at least 24 months. In certain embodiments, the implant 10 can be removed prior to release of the entirety of the pharmaceutical agent, for example if the subject experiences undesired side effects.

When the implant 10 is in an aqueous environment (e.g., the vitreous of the eye), the pharmaceutical agent will diffuse from the core 12 out of the aperture 14 and optionally second aperture 24. In certain embodiments, the implant releases an initial burst of the pharmaceutical agent followed by a substantially zero-order kinetic release of the pharmaceutical agent. The initial burst releases the pharmaceutical agent at a rate that is greater than the rate of the substantially zero-order kinetic release of the pharmaceutical agent. In certain embodiments, the substantially zero-order kinetic release occurs over at least about 30 days, over at least about 60 days, over at least about 90 days, over at least about 120 days, over at least about 180 days, over at least about 210 days, at least 360 days, or at least 720 days.

In some embodiments of the implant 10 disclosed herein, the implant 10 provides a release rate of the drug from about 70 μg/day to about 0.2 μg/day or from about 70 μg/day to about 0.5 μg/day.

The implant body 14 can be sized as needed for the desired length of deliver of the pharmaceutical agent. In certain embodiments, the implant body 14 has a length of about 2 mm to about 10 mm, about 2 mm to about 7 mm, or about 2 mm to about 5 mm. In certain embodiments, the implant body has a diameter of about 0.1 mm to about 0.5 mm or about 0.1 mm to about 2.0 mm. For delivery, the implant 10 is preferably sized to pass through a needle smaller than a 20-gauge needle (e.g., a 20-30 gauge needle), even more preferably smaller than a 22-gauge needle (e.g., a 22-30 gauge needle). For systemic applications the implant can be sized to pass through a 12 or 14 gauge needle. It will be appreciated that the range of needle sizes is exemplary only, and that the systems described herein may be used to manufacture injectable devices for use with larger or smaller needles than those specifically recited above. It should further be appreciated that the term “injectable devices” Of “injectable implant” as used herein, does not refer strictly to devices that are injectable using only hypodermic needle sizes described above. Rather, the term is intended to be construed broadly, and may include devices that are administered through an arthroscope, catheter, or other medical device. Similarly, the terms “inject” and “injected” are meant to include administration by means other than via hypodermic needle, such as by arthroscope, catheter, or other medical device. Accordingly, the term “needle size” can mean the size of the incision through which the device is inserted.

The pharmaceutical agent can have a solubility less than about 2 mg/ml or preferably less than about 1 mg/ml. In certain preferred embodiments, the pharmaceutical agent is islatravir or K8. In certain embodiments, the pharmaceutical agent has moderate-low solubility. In certain embodiments, “moderate-low solubility” indicates a solubility less than about 2 mg of compound per 1 ml (of water at a temperature of about 25° C. as measured by procedures set forth in the 1995 USP).

Examples of compounds having a low solubility include, but are not limited to, immune response modifiers such as cyclosporine A and FK 506, corticosteroids such as dexamethasone, fluocinolone acetonide and triamcinolone acetonide, anti-parasitic agents such as atovaquone and chloroquine, anti-glaucoma agents such as brimonidine, antibiotics including erythromycin and ciprofloxacin, differentiation modulators such as retinoids (e.g., trans-retinoic acid, cis-retinoic acid and analogues), anti-viral agents such as islatravir including high molecular weight low (10-mers) low solubility anti-sense compounds, siRNA molecules, anti-cancer agents such as BCNU, methotrexate, regorafenib, axitinib, crenolanib, cabozantinib, gefitinib, erlotinib, sorafenib, sunitinib, dasatinib, nintedanib, ponatinib, linifanib, and afatinib, and nonsteroidal anti-inflammatory agents such as indomethacin, mefenamic acid, and flurbiprofen, and low solubility derivatives of nucleoside reverse transcriptase inhibitors such as K8.

Bioerosion of the polymeric material of the implant body 14, the core 12, and the coating polymer (e.g., layer 21) can occur through slow dissolution of the polymer in the aqueous media. Water-soluble polymers which are also compatible with the human tissue are known and include, but are not limited to, polyvinyl alcohol (PVA), hydroxypropyl cellulose, polyacrylic acid, hydroxypropylmethyl cellulose, carboxymethyl cellulose, and hydroxyethyl cellulose. Such polymers are commercially available or can be made by methods known in the art. Preferred water-soluble biocompatible polymer is PVA. Water-soluble PVA has been shown to be non-toxic when used in eye implants or eye injections (see, e.g., W. Morton Grant, Joel S. Schuman Toxicology of the Eye, 4^{th} Edition, Springfield, IL: Charles C Thomas Pub., Ltd, 1993, V. 1, p. 1189).

The solubility of a polymer in water depends on multiple parameters, including, but not limited to, molecular weight of the polymer, its degree of crosslinking, the nature and number of pendant groups, and its degree of crystallinity.

One of the parameters controlling the solubility of PVA is its degree of hydrolysis. PVA is prepared by hydrolysis of pendant acetate groups in a polyvinyl acetate precursor. As used herein, the “degree of hydrolysis” means the mole percent of pendant alcohol groups present in PVA out of the total number of pendant groups.

Another parameter controlling the solubility of PVA is its degree of crystallinity. Samples of PVA with higher degree of crystallinity display lower water solubility. The degree of crystallinity of PVA can be increased to a desired extent by heating the polymer for a certain period of time to a specific temperature (see, e.g., Peter R. Byron, Richard N. Dalby, (1987). Effects of Heat Treatment on the Permeability of Polyvinyl Alcohol Films to a Hydrophilic Solute. Journal of Pharmaceutical Sciences, 76 (1), 65-67). Alternatively, the degree of PVA crystallinity can be increased by subjecting the material to multiple freeze-thaw cycles.

Suitable matrix and coating polymers should be non-toxic and should withstand sterilization of the pellet (e.g., by gamma radiation, e-beam, heat/steam and/or ethylene oxide gas) without significant degradation.

As used herein, the “polydispersity index” is a ratio that describes the homogeneity of the particle size distribution of a system. A small value, e.g., less than 0.3, indicates a narrow particle size distribution.

The compounds that may be employed in the practice of the present invention should be in moderate-low solubility form, e.g., a less-polar free-base or free-acid form rather than a salt, and/or a crystalline form rather than a more-soluble amorphous form, as needed to provide a desired release rate. Reference may be made to any standard pharmaceutical textbook for the procedures to obtain a moderate-low solubility form of a drug.

The method for treating a mammal to obtain a desired local or systemic physiological or pharmacological effect includes injecting the implant 10 of the present invention into the mammal. In addition, one or more of the implants 10 may be administered at one time (e.g., in one injection), or more than one agent may be included in the core. Intravitreal injection is a minimally invasive procedure that has become an effective intervention in the management of numerous eye diseases. The procedure may include an implant 10 being directly injected into the vitreous cavity of the eye of a patient. The procedure bypasses anatomical barriers in the eye.

Intravitreal injectors are disclosed, for example, in the following U.S. Patents: U.S. Pat. Nos. 7,678,078, 8,133,273, 8,287,494, 8,945,214, 9,421,129, 9,693,893, and 9,974,645, each of which is hereby incorporated by reference in its entirety.

Implants 10 of the present invention are particularly suitable for direct injection into the eye. In certain embodiments, the implant 10 may be injected in the vicinity of a patient's eye as either an intraocular or periocular injection. For example, the implant 10 can be injected into the vitreous humor, or injected into the anterior chamber. In some embodiments the implant 10 is administered by intraocular injection (e.g., localized intraocular therapy); intravitreous injection; subretinal injection; suprachoroidal injection; episcleral injection; sub-Tenon's injection; retrobulbar injection; or peribulbar injection.

Implants 10 of the present invention can be administered by subcutaneous injection, intratumoral injection, intracranial injection, or intraarticular injection, for example. For example, the implant 10 can be injected be injected under the skin of a patient's arm, leg, or torso. In certain preferred embodiments, an implant 10 comprising islatravir can be injected subcutaneously.

These methods of administration and techniques for their preparation are well known by those of ordinary skill in the art. Techniques for their preparation are set forth in Remington's Pharmaceutical Sciences.

The presently-disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

In one aspect, the present disclosure relates to an implant 10 shaped and sized for injection, consisting essentially of a plurality of drug particles pressed into a pellet, wherein the drug has a solubility in water less than about 100 μg/mL at about 23° C. In preferred embodiments, the implant 10 does not include any polymeric matrix or polymeric coating.

In some embodiments of the implant 10 disclosed herein, the device has a largest dimension from about 1 mm to about 30 mm or about 1 mm to about 10 mm. The implant 10 may have an aspect ratio from about 1.5 to about 25.

In certain embodiments, the present disclosure relates to a method for treating a mammalian organism to obtain a desired physiological or pharmacological effect comprising administering the drug delivery device to a mammalian organism in need of such treatment.

Certain embodiments of this invention are described herein. Of course, variations, changes, modifications and substitution of equivalents of those embodiments will be apparent to those of ordinary skill in the art upon reading the foregoing description. Skilled artisans may employ such variations, changes, modifications and substitution of equivalents as appropriate, and so may practice the invention otherwise than specifically as described herein. Those of skill in the art will readily recognize a variety of non-critical parameters that may be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Definitions

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

As used herein, the abbreviations for any protective groups and compounds, are, unless indicated otherwise, in accord with their common usage, recognized abbreviations, or the IUPAC-IUB Commission on Biochemical Nomenclature (see (1972) Biochemistry, 11(9), 1726-1732).

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method. As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

With regard to administering the formulation, the term “administering” refers to any method of providing a composition and/or pharmaceutical composition thereof to a subject. Such methods are well known to those skilled in the art and include, but are not limited to, intravitreous administration, including via intravitreous sustained implants, intracameral (into anterior chamber) administration, suprachoroidal injection, subretinal administration, subconjunctival injection, sub-Tenon's administration, peribulbar administration, transscleral drug delivery and the like. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent, temporary, lasting, or transitory, that can be associated with the administration of the pharmaceutical composition.

The term “subject” can refer to a human or non-human subject (e.g., primate, horse, pig, rabbit, dog, sheep, goat, cow, cat, guinea pig, rodent, and non-mammals). The term “subject” does not denote a particular age or sex. Thus, adult and newborn subjects, as well as fetuses, whether male or female, are intended to be covered. The term “subject” includes human and veterinary subjects.

The term “sustained release” refers to a drug-containing formulation or dosage form, e.g., an implant that provides for gradual release of a pharmaceutical agent over an extended period of time.

“Substantially impermeable” as used herein refers to inhibiting diffusion therethrough by at least an order of magnitude, two orders of magnitude, or preferably at least three orders of magnitude.

“Bioerode” or “bioerosion”, as used herein, refers to the gradual disintegration or breakdown of a solid structure or a material over a period of time in a biological system into molecular building blocks, by one or more physical or chemical processes, for example, dissolution by solubilization, emulsion formation, or micelle formation.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Tubes of polyimide (MicroLumen) were obtained, cut to 10 cm in length and fixed to the end of a syringe. Separately a quantity of the drug K8 (synthesis of K8 is described in US20180044327, published Feb. 15, 2018, the entirety of which is hereby incorporated herein by reference) was melted on a heating pad and drawn into the polyimide tube. After cooling, the tube was cut to length (e.g., 2.5 mm, 5 mm, etc.). A similar approach can be used for making K8 implants using PLGA tubes (Biogeneral.).

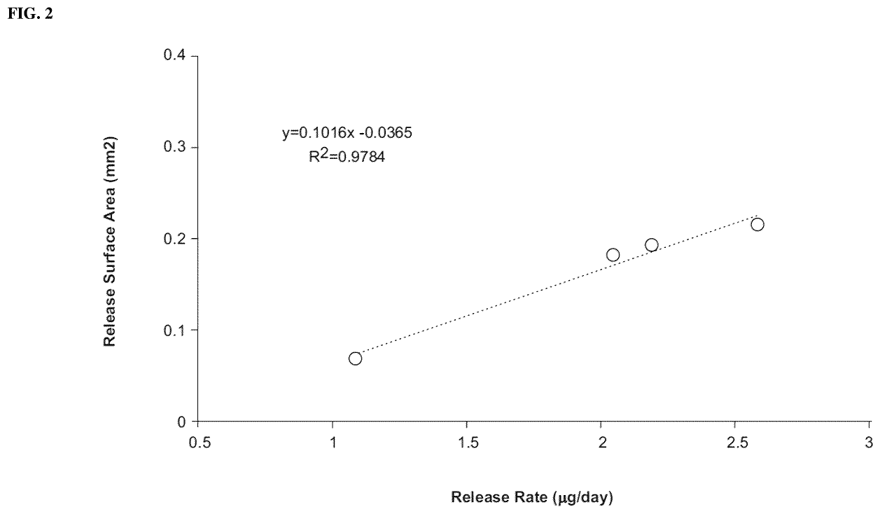

The implants show a steady state release of material, as shown in FIGS. 1B and 2 and as illustrated in FIG. 3.

TABLE 1

| | | | Surface | | | |
|---|---|---|---|---|---|---|
| | | Diameter Ø | Area | RR | Plateau | Linear |
| | Tube | (inch/mm) | (mm²) | (μg/day) | Duration | Regression R² |
| 1 | PLGA tube | 0.008/0.21 | 0.0692 | 1.09 | Day 25-57 | 0.999 |
| | (3.5 mm long) | | | | | |
| 2 | Polyimide tube | 0.0135/0.34 | 0.1815 | 2.05 | Day 25-95 | 0.997 |
| | (3.5 mm long) | | | | | |
| 3 | PLGA tube | 0.0138/0.35 | 0.1923 | 2.19 | Day 40-100 | 0.996 |
| | (3.5 mm long) | | | | | |
| 4 | Polyimide tube | 0.0145/0.37 | 0.2149 | 2.58 | Day 30-120 | 0.996 |
| | (5.0 mm long) | | | | | |

### Example 2

Tubes of polyimide (MicroLumen) were obtained, cut to 10 cm in length and fixed to the end of a syringe. Separately a quantity of the drug K8 (synthesis of K8 is described in US20180044327, published Feb. 15, 2018, the entirety of which is hereby incorporated herein by reference), was melted on a heating pad and drawn into the polyimide tube. After cooling, the tube was cut to length (e.g., 2.5 mm, 5 mm etc.). A similar approach can be used for making K8 implants using PLGA tubes (Biogeneral.).

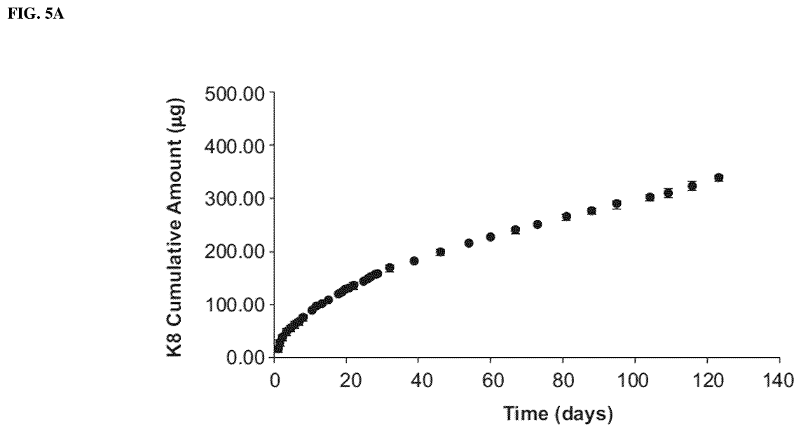

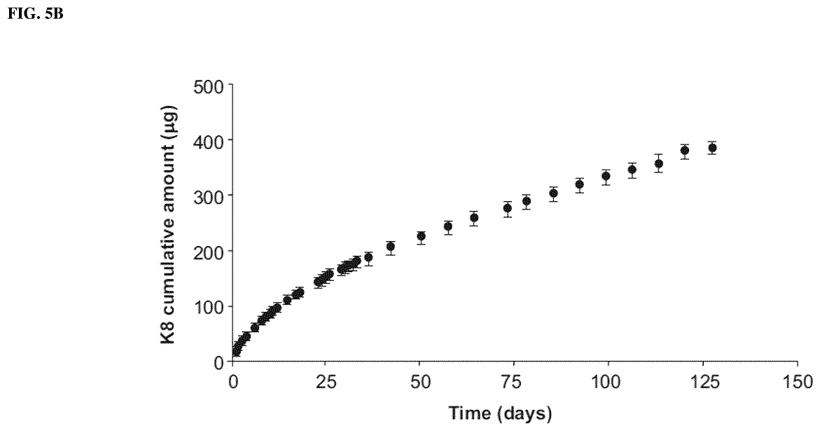

FIGS. 5A-B show 100% release of K8 from two similar implants. As can be seen, after the initial burst release continues at a relatively constant rate until all the drug has been released. The two implants are composed of a K8 core (i.e., no other material) in either a polyimide tube (non-erodible) or a PLGA tube (bioerodible).

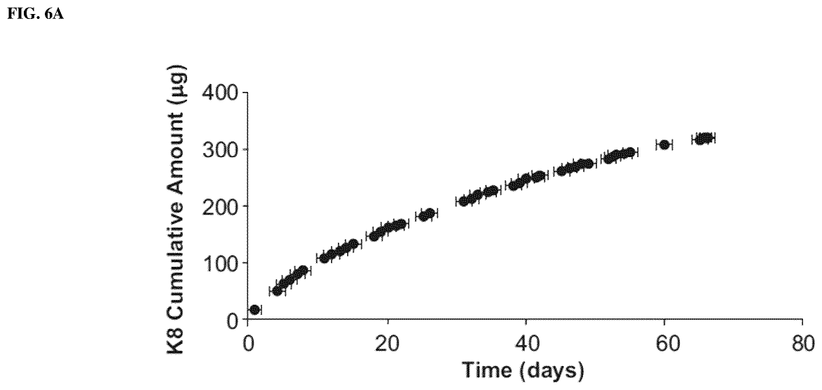

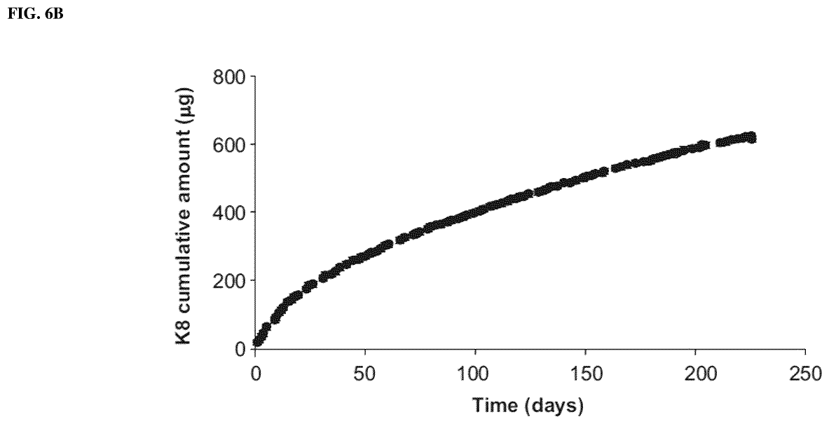

The duration the constant phase of drug release is governed by the length of the tube although it is not directly proportional to the length (as the initial burst will be the same regardless of the length of the tube). FIGS. 6A-B show a 2.5 mm long tube containing 320 μg of drug provides sustained release for approximately 60 days while a tube 5 mm in length containing 610 μg provides sustained release for approximately 220 days.

In the above experiments in-vitro release was measured by immersing the implant into a PBS solution (pH 7.4) at 37° C. Samples were taken periodically and assayed by HPLC and the entire medium replaced periodically to maintain sink conditions. Graphs shown are cumulative release versus time.

The release rate was determined in-vivo by injecting implants into the vitreous of rabbits. Animals were sacrificed at days 28, 56, and 120 and their eyes removed for analysis. Vitreous and drug concentrations were measure and the amount of residual drug in the implants determined (Table 2). The amount of drug released in the eye was calculated (by subtracting the amount of drug remaining from the initial amount of drug in the implant). FIG. 7 shows a plot of the cumulative release in-vitro versus the actual release in-vivo and shows excellent in-vitro to in-vivo correlation.

TABLE 2

| | | Estimated K8 | Measured K8 | K8 level in |
|---|---|---|---|---|
| | Explants K8 | Released in | released in | vitreous |
| Time | Residue | vivo | vitro | (ng/ |
| (days) | (μg) N = 4 | (μg) | (μg) N = 4 | ml)N = 4 |
| Day 28 | 407.8 ± 20.2 | 193.2 | 195.7 ± 5.4 | 575 ± 334 |
| Day 56 | 328.1 ± 6.2 | 272.9 | 289.5 ± 2.3 | 128 ± 25 |
| Day 120 | 182.1 ± 22.1 | 418.9 | 440.4 ± 3.4 | 122 ± 40 |

Note:
On Day 120, 4 samples of Retina/Choroid were separated collected and analyzed. The result is 87.78 ± 34.84 ng/ml

After drug has been released the implant body can fully erode. Polymers such as PLGA are available in different erosion rates. Polymers such a PLGA, which undergo “bulk” erosion are well suited to these systems.

### Example 3

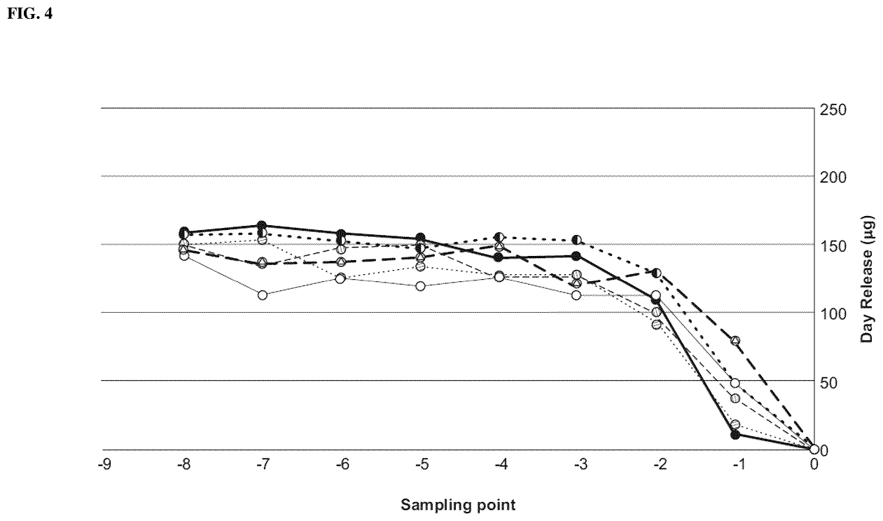

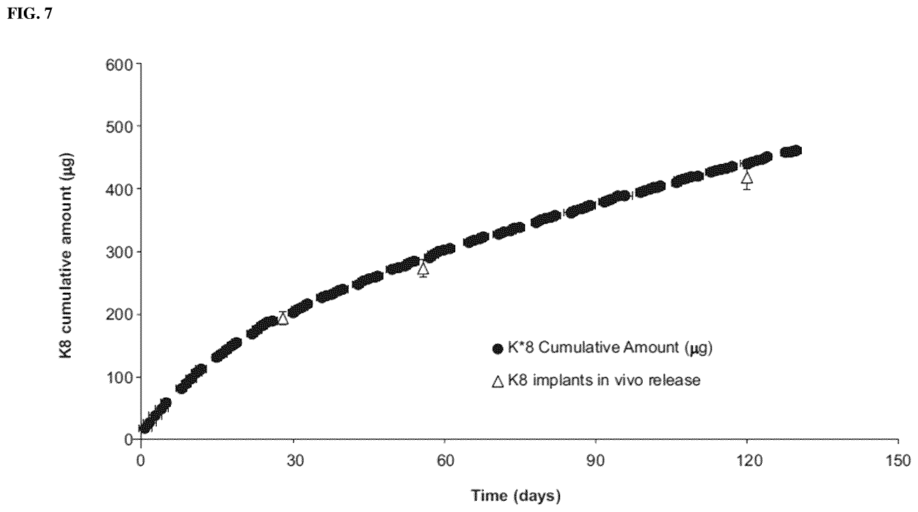

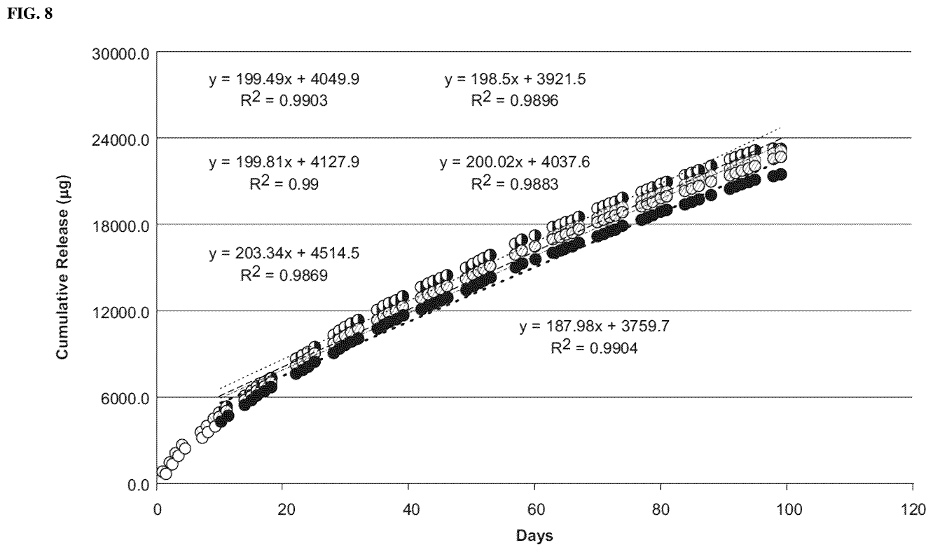

EFdA (islatravir) was mixed with an equal weight of 5% aqueous solution of polyvinyl alcohol (EMD Chemicals), wet granulated and dried. The particles were then compressed in a 2 mm tablet die to form 24 mg pellets (containing 4% PVA). Pellets were dipped in 5% PVA solution and inserted into tubes of polyimide 2 mm in diameter. Then the dry implants were heated at 105° C. for 2 hours. These implants gave complete release over approximately 100 days and followed the same pattern as the much smaller K8 implants with an initial burst followed by a more constant release until full depletion (FIG. 8). As the implants became depleted there was an extremely fast decline in release (FIG. 4), similar to that of the K8 implants.

The release of similar EFdA implants (2 mm in diameter, 5% PVA, heated at 105° C.) was tested in buffer without first placing them in polyimide tubes. The implants fully released EFdA and disintegrated within 7 days.

PVA, at the low concentration used, does not significantly slow down the release of EFdA and does not provide physical stability. When the EFdA/PVA implants are in the tube, however, the matrix does retain the EFdA in the tube and thus ensures the surface area available for dissolution is maintained.

### Example 4

### EFdA Implants-In Vitro Release

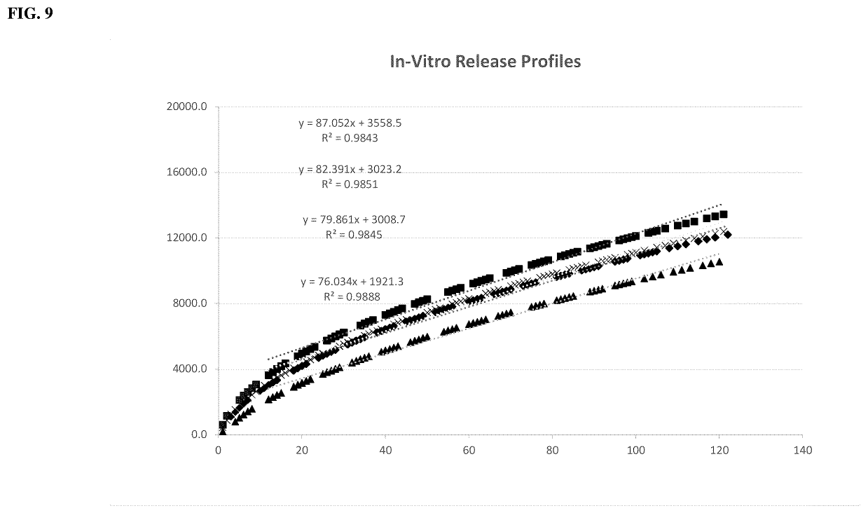

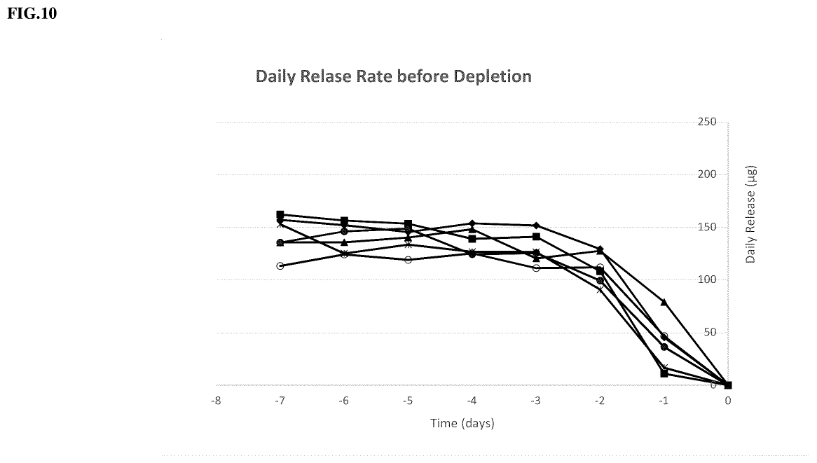

Implants were placed in phosphate buffered saline (pH 7.4), typically 1 -10 ml in a microcentrifuge tube or covered test tube. These were placed in a water bath at 37° C. Daily samples were removed for analysis by HPLC and replaced with fresh buffer. The entire buffer was regularly replaced to ensure sink conditions were maintained. Graphs were plotted of cumulative release versus time to determine the release rate (FIG. 9). When determining the tail-off period, release rates were measured until the amount released is below the level of detection. The daily release was then plotted on preceding days to determine the daily release rate before depletion (FIG. 10). The x-axis is days prior to termination, i.e. −1, −2 etc. This was necessary because a simple average would give a false assessment if implants became depleted on different days. As a somewhat morbid example, if there were 10 people who died (let's say over 10 days) and one measured their temperatures, the average temperature of the group would suggest that temperature steadily declines in the last 10 days of life. If one measure temperature in the 10 days prior to each person's death the results would be different.
The developed EFdA implant is a compressed pellets inserted in an impermeable tube, and both ends open.
Implants in PLGA tube (bio-degradable) showed a similar in vitro release profile compared to implants in polyimide tube (non-degradable).
If the release surface area is equivalent to 3.14 mm², the daily release rate at steady state phase is around 75 μg/day (FIG. 9).
After a brief burst effect release is relatively constant in-vitro (FIG. 9) and with this type of device release is more or less constant until the device becomes fully depleted at which point release falls off very quickly (FIG. 10).
The implants showed a short tail and depleted completely in a few days in vitro (FIG. 10).
With both ends open for release, 1.5 mm diameter implants, 5 mm long can last for 3 month and 13 mm long can last for 12 months.

### Example 5

### EFdA Implants in Rats

### Study Design:

EFdA implants (in PLGA tube) of two different sizes are tested in this study:
Implant #1:
   A short duration implant targeted for three-month release.
   This study arm shows the tail effect in a short duration of three months.
Implant #2:
   A potential clinical dose implant targeted for 12-month release. This study arm shows PK and tail effect in a long duration of 12 months.
      Blood samples are collected according to the schedule and analyzed for EFdA plasma concentration to evaluate:
The burst effects
Steady state kinetics of release by measuring plasma levels (and residual drug content of explanted devices)
Tail effect.
PLGA tube integrity in 12 months duration

Administration route: Once time subcutaneous Implantation

| | | | 3-month implant | 12-month implant |
|---|---|---|---|---|
| | | | 6 × rat | 12 × rat |
| | | | Implant dimension: | Implant dimension: |
| | | Material | 1.5 mm ID × 5.0 mm L, | 1.5 mm ID × 13 mm L |
| Time | | sampled | 12 mg API | 29 mg API |
| Pre-dose | | Blood | (6) bloods only, no sacrifice | (6) bloods only, no sacrifice |
| 1 hour | | Blood | (6) bloods only, no sacrifice | (6) bloods only, no sacrifice |
| 3 hours | | Blood | (6) bloods only, no sacrifice | (6) bloods only, no sacrifice |
| 5 hours | | Blood | (6) bloods only, no sacrifice | (6) bloods only, no sacrifice |
| 8 hours | | Blood | (6) bloods only, no sacrifice | (6) bloods only, no sacrifice |
| 1 day | | Blood | (6) bloods only, no sacrifice | (6) bloods only, no sacrifice |
| 3 day | | Blood | (6) bloods only, no sacrifice | (6) bloods only, no sacrifice |
| 7 day | | Blood | (6) bloods only, no sacrifice | (6) bloods only, no sacrifice |
| 14 day | | Blood | (6) bloods only, no sacrifice | (6) bloods only, no sacrifice |
| 1 month | | Blood | (6) bloods only, no sacrifice | (6) bloods only, no sacrifice |
| 2 months | | Blood | (6) bloods only, no sacrifice | (6) bloods only, no sacrifice |
| 3 M | 70 days | Blood | (6) bloods only, no sacrifice | (6) bloods (2) Implant remove |
| | 80 days | Blood | (6) bloods only, no sacrifice | |
| | 90 days | Blood/ | (6) bloods/sacrifice | |
| | | Implant | | |
| | | remove | | |

| | | |
|---|---|---|
| 4 months | Blood | (6) bloods only, no sacrifice |
| 5 months | Blood | (6) bloods only, no sacrifice |
| 6 months | Blood, | (6) blood (2) Implant remove |
| | implant | |
| | remove | |
| 7 months | Blood | (6) bloods only, no sacrifice |
| 8 months | Blood | (6) bloods only, no sacrifice |
| 9 months | Blood, | (6) bloods (2) Implant remove |
| | implant | |
| | remove | |
| 10 months | Blood | (6) bloods only, no sacrifice |
| 11 months | Blood | (6) bloods only, no sacrifice |
| 12 months | Blood, | (6) blood/sacrifice |
| | implant | |
| | remove | |
| 13 months* | Blood, | Potential study extension |
| | implant | |
| | remove | |

Total rats' number: 6 (3 M) + 12(12 M) + 2 (extra) = 20

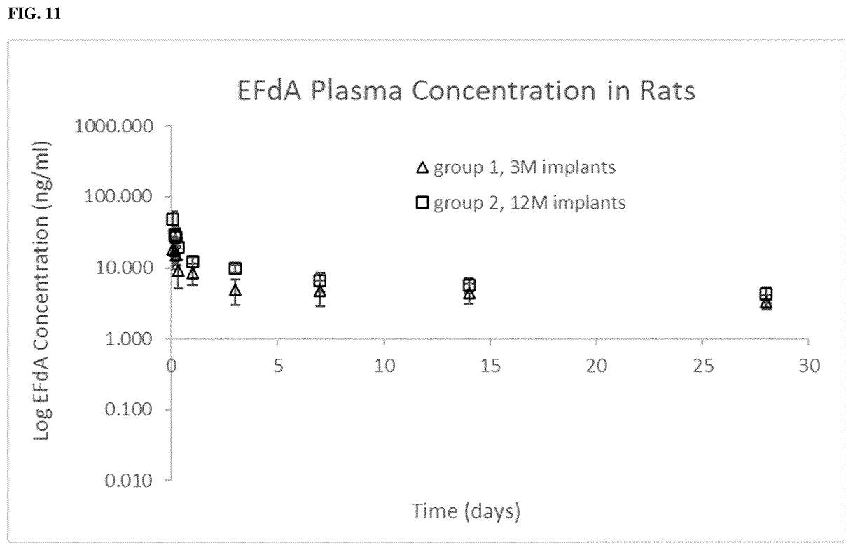

### Batch Sample Results (FIG. 11):

1. There was an initial burst in the first day and continue to decreasing in the first week. The plasma concentration was getting stable from day 7 to day 30 with the EFdA concentration in the range of 4-6 ng/ml.
2. The short and long implants showed similar plasma concentration even though the long duration implants had more than twice as much drug and the total surface area (including the sides of the tubes) was much larger. The reason for this is that for both implants the ends of the tubes were open to diffusion as they had the same diameter their release rate was the same.
3. The dosing went smooth, and animals tolerated well by observation
4. The bioanalytical method has been established for EFdA in rat's plasma
   This work demonstrates the advantages of the new implant, for example:
1) Release rate is relatively constant after a short burst effect
2) Release rate is controllable (in this case by controlling the diameter of the tube)
3) Release is relatively constant until the device is fully depleted (no tail-off effect)
4) By inference, duration is easily controlled (by changing the length)
5) By inference, these implants are very easy to manufacture since no rate limiting membrane needs to be applied to the ends of the tube (and a membrane would require a tricky manufacturing process as one has to ensure the membrane is of reproducible thickness and the manufacturing process must be validated)

1. A drug delivery implant shaped and sized for injection, the implant comprising:
a core comprising a pharmaceutical agent, and
a cylindrical implant body configured to extend longitudinally around and circumferentially contact a longitudinal surface of the core, and that defines an aperture disposed on a longitudinal surface of the implant body to expose a first surface of the core,
wherein, when the implant is placed in an aqueous environment, the pharmaceutical agent diffuses from the first surface of the core through the aperture of the implant body.

2. The implant of claim 1, wherein two or more apertures on the longitudinal surface expose surfaces of the core.

3. A drug delivery implant shaped and sized for injection, the implant comprising:
a core comprising a pharmaceutical agent, and
a cylindrical implant body configured to extend longitudinally around and circumferentially contact a longitudinal surface of the core, and that defines an aperture at a first end of the implant body to expose a first surface of the core,
wherein, when the implant is placed in an aqueous environment, the pharmaceutical agent diffuses from the first surface of the core through the aperture at the first end of the implant body.

4. The implant of claim 1, wherein the pharmaceutical agent has a solubility less than about 2 mg/ml or less than about 1 mg/ml.

5-8. (canceled)

9. The implant of claim 1, wherein the core comprises drug particles and the pharmaceutical agent is present in the drug particles.

10. The implant of claim 9, wherein the core comprises a pellet comprising the drug particles and optionally a polymeric material.

11. The implant of claim 1, wherein the core comprises a matrix that comprises the pharmaceutical agent and a polymeric material.

12. The implant of claim 11, wherein the polymeric material is present at less than about 10% w/w of the core, less than about 5% w/w of the core, or less than about 2% w/w of the core.

13-14. (canceled)

15. The implant of claim 11, wherein the polymeric material does not substantially affect the rate of release of the pharmaceutical agent.

16. The implant of claim 11, wherein the polymeric material is PVA.

17. The implant of claim 1, wherein the pharmaceutical agent is islatravir or K8.

18-19. (canceled)

20. The implant of claim 1, wherein the implant body comprises polyimide, PLGA, PCL, or PLA.

21-23. (canceled)

24. The implant of claim 1, wherein the implant body has a length of about 1 mm to about 30 mm, or about 2 mm to about 7 mm, or about 2 mm to about 5 mm, or about 0.1 mm to about 0.5 mm, or about 0.1 mm to about 2 mm.

25-28. (canceled)

29. The implant of claim 1, wherein when the implant is placed in an aqueous environment, the implant releases an initial burst of the pharmaceutical agent followed by substantially zero-order kinetic release of the pharmaceutical agent, wherein the initial burst releases the pharmaceutical agent at a rate that is greater than the rate of the substantially zero-order kinetic release of the pharmaceutical agent.

30. The implant of claim 29, wherein the substantially zero-order kinetic release occurs over at least about 30 days, at least 60 days, at least 90 days, at least 120 days, at least 180 days, at least 210 days, at least 360 days, or at least 720 days.

31-37. (canceled)

38. A drug implant comprising a core, wherein the core comprises a pharmaceutical agent having a solubility less than about 2 mg/ml, less than about 1 mg/ml, or about 0.2 to about 0.7 mg/ml.

39. The drug implant of claim 38, wherein core comprises a polymer present at less than about 5 wt % of the core.

40-41. (canceled)

42. The implant of claim 38, wherein the pharmaceutical agent is K8 or islatravir.

43. A method for treating a subject in need thereof, comprising implanting an implant of claim 1.

44. (canceled)

45. The method of claim 43, wherein implanting comprises administering the implant by subcutaneous injection, intratumoral injection, intracranial injection, or intraarticular injection, or intraocular injection, e.g., intravitreous injection; subretinal injection; episcleral injection; sub-Tenon's injection; retrobulbar injection; or peribulbar injection.

46. (canceled)
